(19) United States
(12) Patent Application Publication
(10) Pub. No.: US 2024/0341897 A1
(43) Pub. Date: Oct. 17, 2024

(54) TORQUE-LIMITING SLIP-CLUTCH SYSTEM WITH MODULAR DISKS

(71) Applicant: ECA Medical Instruments, Inc., Thousand Oaks, CA (US)

(72) Inventors: Alexander Arazawa, Thousand Oaks, CA (US); Navid Mahpeykar, Thousand Oaks, CA (US); Andre Comella, Thousand Oaks, CA (US); Tommy Tak, Thousand Oaks, CA (US)

(21) Appl. No.: 18/634,017

(22) Filed: Apr. 12, 2024

Related U.S. Application Data
(60) Provisional application No. 63/458,779, filed on Apr. 12, 2023.

(51) Int. Cl. A61B 90/00; F16D 7/02

(52) U.S. Cl. CPC A61B 90/03; F16D 7/024; A61B 2090/031; F16D 2200/0004; F16D 2200/0065

(57) ABSTRACT

The present disclosure relates generally to systems and methods to limit the torque of tools and devices, and specifically, to single-use or disposable, low-cost slip clutch systems and methods with modular disks that limit the output torque of manual or power-driven medical drivers.

### REFERENCE TO RELATED APPLICATIONS

This application claims the priority benefit of U.S. Provisional Patent Application No. 63/458,779, filed on Apr. 12, 2023 and entitled “TORQUE-LIMITING SLIP-CLUCTH SYSTEM WITH MODULAR DISKS,” which is fully incorporated by reference herein in its entirety. Applicant notes that the title of the present application is identical to the aforementioned provisional patent application, but for the correction of a minor typographical error in the spelling of “clutch.”

### BACKGROUND OF THE DISCLOSURE

### Field of the Disclosure

The present disclosure relates generally to manual and power-driven tools. More specifically, this disclosure relates to systems and methods for limiting the torque of tools and devices, and specifically, to single-use or disposable, low-cost slip clutch systems and methods with modular disks that limit the output torque of manual or power-driven medical drivers.

### Description of the Related Art

Drivers with integrated torque-limiting, as well as drivers coupled to a torque-limiting attachment are commonly used throughout the medical industry. Medical professionals often use manual or power-driven drivers to secure threaded fasteners to spinal or orthopedic implants. The medical drivers have integrated torque-limiting functions or are coupled with a torque-limiting attachment to apply a precise amount of torque to the threaded fasteners, which prevents over-torquing and under-torquing of the threaded fasteners.

In a surgical context, there is little room for error, and these drivers must apply a precise amount of torque. Reusable torque-limiting drivers and attachments require constant recalibration to ensure that the driver imparts a precise torque. Reusable drivers logically also require cleaning and sterilization after every use.

Disposable or single-use torque-limiting drivers and attachments are an alternative to reusable drivers. Disposable torque-limiting drivers and attachments are generally for used low torque applications due to the strength limitations of typical low-cost engineering materials and manufacturing methods.

Accordingly, a single-use or disposable torque-limiting system that can be both manufactured at low cost and allows for higher torque thresholds would be advantageous.

### SUMMARY OF THE DISCLOSURE

Some embodiments of a torque-limiting slip-clutch system according to the present disclosure comprise a top clutch disk and an opposing bottom clutch disk, each configured with a plurality of lobes; an elastic component; and a clutch carrier configured to receive said elastic component, wherein said clutch carrier is configured to interface with said top clutch.

This has outlined, rather broadly, the features and technical advantages of the present disclosure so that the detailed description that follows may be better understood. Additional and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims.

The novel features, which are believed to be characteristic of the disclosure, both as to its organization and method of operation, together with further features and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 shows a front view of a torque limiting attachment that features an embodiment of a slip-clutch system incorporating features of the present disclosure with the clutch carrier made transparent;

FIG. 2 shows an exploded view of a torque-limiting module of a torque-limiting attachment that includes an embodiment of a slip-clutch system incorporating features of the present disclosure;

FIGS. 3A-3C show top perspective, right side, and bottom perspective views, respectively, of an embodiment of a clutch disk incorporating features of the present disclosure;

FIGS. 4A-4C show top perspective, right side, and bottom perspective views, respectively, of another embodiment of a clutch disk incorporating features of the present disclosure;

FIGS. 5A-5C show top perspective views of other embodiments of clutch disks incorporating features of the present disclosure;

FIG. 6 shows a top perspective view of another embodiment of a clutch disk incorporating features of the present disclosure;

FIGS. 7A-7C show top perspective, left side, and top views of two embodiments of cone-shaped washers (i.e., Bellville washers) for use in embodiments of the present disclosure; and

FIGS. 8A-8E show top perspective, bottom, top, right side, and bottom perspective side views of an embodiment of a clutch carrier incorporating features of the present disclosure.

### DETAILED DESCRIPTION OF THE DISCLOSURE

Described herein are low-cost, disposable or single-use torque-limiting systems and methods comprising modular clutch disks with lobes and a separate clutch carrier that work together with an elastic element (e.g., spring stack) to precisely limit output torque in applications or devices in which precise torque-limiting is needed. Separating the clutch disks from the clutch carrier allows for easier (and thus more cost-effective) manufacturing and higher torque thresholds. The torque-limiting systems and methods disclosed herein may be used in medical drivers or in any other applications or devices where torque-limiting is required.

Slip-clutches are used in a variety of applications, including torque transmission and torque-limiting applications. Clutches typically include first and second members movable relative to one another and a means of providing a selected amount of torsional friction between the two members so that relative motion of the two members occurs only when they are subjected to a torque that exceeds a predetermined “slip” or “break away” torque associated with the clutch.

The torsional friction may be provided by an elastic and/or spring-loaded element, such as a coil spring or slightly angled washers, associated with the two members wherein the elastic component is mounted or connected to one member and arranged to impart a normal force to frictionally engage the other member.

Embodiments of the present disclosure prevent both over-torquing and under-torquing. Over-torquing (i.e., too much force) can result in stripped fasteners, sheared fasteners or driver heads, and/or cold-welded threads, among other issues. Under-torquing can result in an unsecure joint that can loosen and fail over time.

It is understood that when an element is referred to as being “on” another element, it can be directly on the other element or intervening elements may also be present. Similarly, if an element is “attached to,” “connected to,” or similar, another element, it can be directly attached/connected to the other element or intervening elements may also be present. Furthermore, relative terms such as “inner”, “outer”, “upper”, “top”, “above”, “lower”, “bottom”, “beneath”, “below”, and similar terms, may be used herein to describe a relationship of one element to another. Terms such as “higher”, “lower”, “wider”, “narrower”, and similar terms, may be used herein to describe angular and/or relative relationships. It is understood that these terms are intended to encompass different orientations of the elements or system in addition to the orientation depicted in the figures.

Although the terms first, second, etc., may be used herein to describe various elements, components, regions and/or sections, these elements, components, regions, and/or sections should not be limited by these terms. These terms are only used to distinguish one element, component, region, or section from another. Thus, unless expressly stated otherwise, a first element, component, region, or section discussed below could be termed a second element, component, region, or section without departing from the teachings of the present disclosure.

Embodiments of the disclosure are described herein with reference to view illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region and are not intended to limit the scope of the disclosure.

Some embodiments incorporating features of the present disclosure are single-use or disposable. One such torque-limiting slip-clutch system comprises two modular slip-clutch disks with an axial lobe design, an elastic element, and a clutch carrier configured to fit the elastic element.

Embodiments disclosed herein may be utilized in certain torque-limiting attachments. Torque-limiting attachments typically prevent over-torquing and under-torquing in medical devices and may comprise a speed reduction mechanism, a torque-limiting system, and a locking mechanism that allows a manufacturer, or even a user (e.g., a surgeon), to set the output torque limit of the attachment or device. More specifically, embodiments of torque-limiting slip-clutch system disclosed herein may be used in a single-use, power-driven torque-limiting attachment.

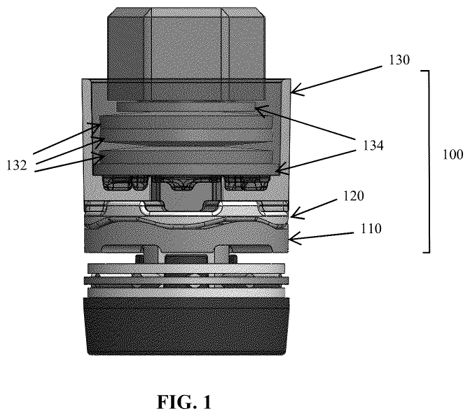

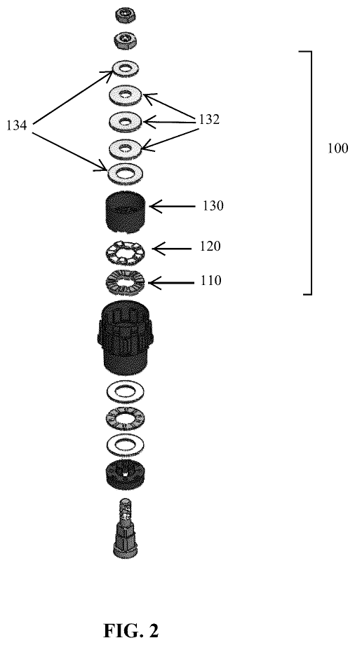

As shown in FIGS. 1 and 2, one embodiment of a torque-limiting slip-clutch system 100 comprises clutch disks 110, 120 and a clutch carrier 130 that contains an elastic element 132. In the embodiment shown, the elastic element is a spring stack created by a plurality Belleville washers 132. However, as one of skill in the art would understand, the elastic element 132 may comprise other or additional elements, such as compression springs, rubber springs, wave springs, and other solutions known in the art.

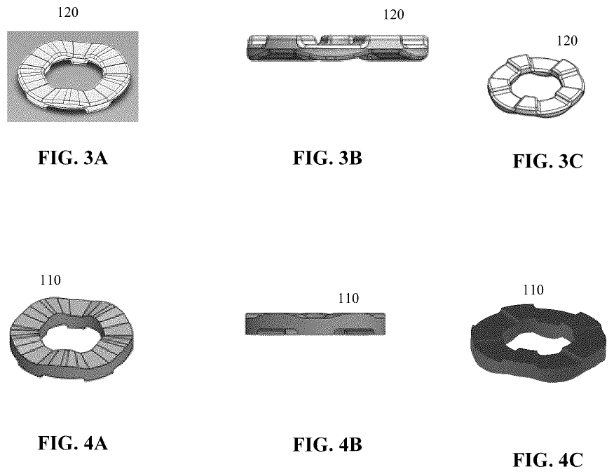

FIGS. 3A-3C and 4A-4C show two embodiments of clutch disks 110, 120 disclosed herein. Both sets of figures show modular clutch disks with axial lobe geometry and, more particularly, five lobes on each clutch disk 110, 120. It is understood, however, that more or fewer lobes are also possible. Similarly, the embodiment shown in FIG. 1 includes two clutch disks 110, 120, but it is understood that two or more clutch disks may be included in the torque-limiting systems disclosed herein.

The clutch disks 110, 120 may be comprised of light-weight, durable materials such as polymer resins, composite-filled polymer resins, metals, metallic alloys, or other suitable materials as would be understood by one of skill in the art. The clutch disks 110, 120 may comprise the same materials or different materials.

The small, modular nature of the slip-clutch components or disks disclosed herein allows for low-cost manufacturing methods, such as injection molding, that result in better material properties, such as injection molding with polymers. Chance of errors or mistakes in manufacturing are also reduced due to the simple disk geometry. These factors, in turn, result in more consistent material properties and geometry of the disks. More consistent and stronger material properties ultimately translate to higher torque values and easier fine-tuning of torque tolerances. These properties also allow for use of various resins and casting alloys to achieve higher torque thresholds.

In some embodiments (not pictured), needle bearings, thrust washers, or other suitable structures are used to provide axial load isolation to make output torque independent of applied axial load.

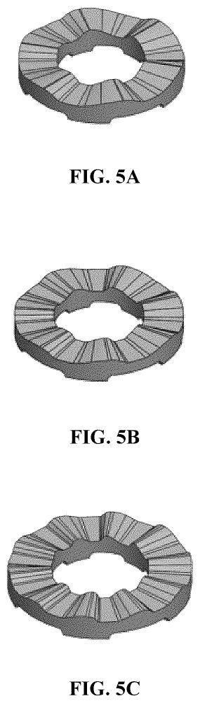

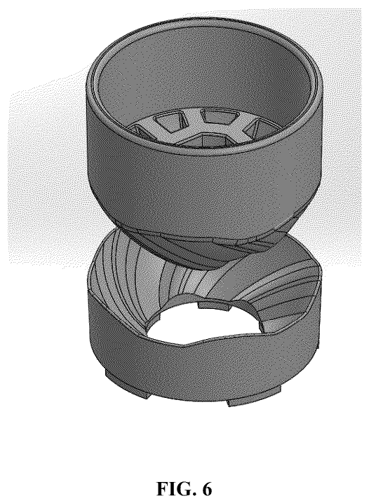

FIGS. 5A-5C and 6 show other embodiments of clutch disks that may be used in torque-limiting systems disclosed herein. As shown in FIGS. 5A-5C, the clutch disk may have differing numbers of lobes. FIGS. 5A-5C show clutch disks with 6, 7, and 10 lobes, respectively. FIG. 6 shows yet another alternative embodiment of a clutch disk showing the clutch disks with a conical and three-dimensional surface design to increase contact surface area of the lobes.

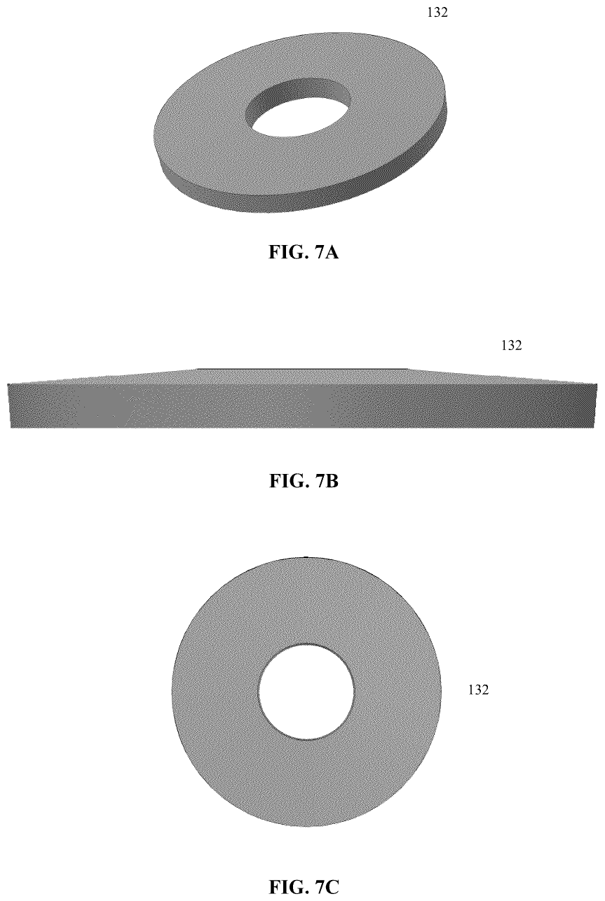

Various views of an embodiment of a coned disk or cupped spring washer 132, commonly referred to as a Belleville washer, are shown in FIGS. 7A-7C. The disks shown do not lie flat as they feature a conical shape in which the inner portion is offset from than the lower portion in the axial direction. Like conventional washers, the Belleville washers shown in FIGS. 7A-7C may have a center hole through which a fastener, such as a bolt, may be placed. When stacked with other Belleville washers, they create a stiff spring or elastic element.

In one embodiment of the slip-clutch torque-limiting system 100, the elastic element is a spring stack comprising at least two flat washers 134 and, in between the flat washers, is at least one Belleville washers 132. While no specific number of flat washers 134 or Belleville washers 132 is required in embodiments incorporating features of the present disclosure, in one particular embodiment, the elastic element comprises at least one Belleville washer 132. It is understood that the elastic member may be implemented using various other structures, such as a stiff coil spring, a diaphragm spring, or other suitable elastic structures. The elastic element is housed in a clutch carrier 130, which is then configured adjacent to the slip-clutch disks, as shown in FIGS. 1 and 2.

When the counter-torque from a driver or other device applying counter-torque exceeds the torque provided by the two modular slip-clutch disks 110, 120 and the spring stack 134, the lobes of the slip-clutch disks slip past one another displacing the spring stack 134, and thereby limiting the output torque applied by the driver or device.

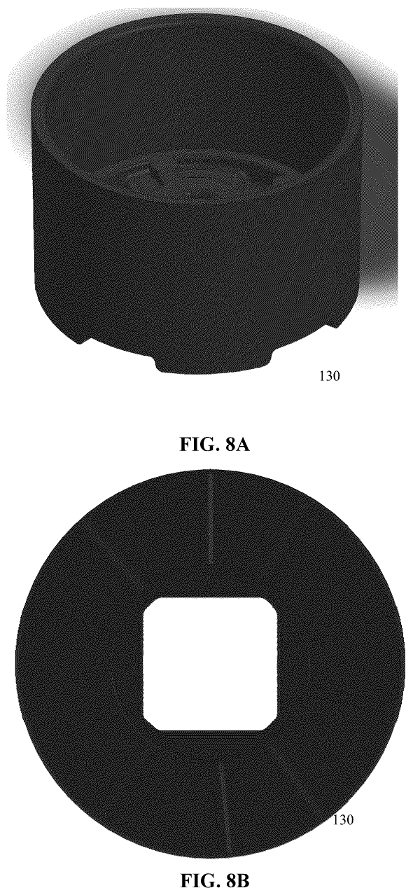

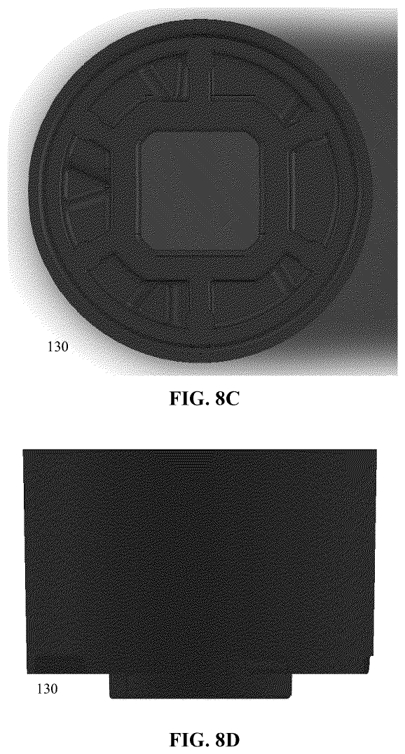

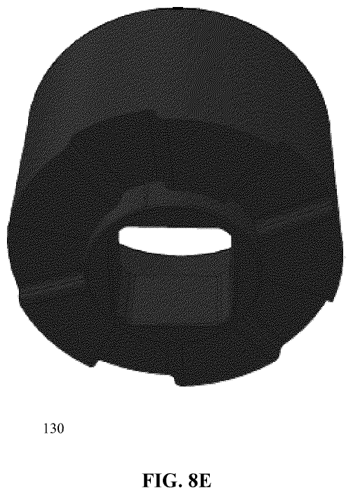

FIGS. 8A-8E show various views of an embodiment of a modular clutch carrier 130. The clutch carrier 130 may be made of many different materials, such as, for example, polymer resins, composite-filled polymer resins, metals, metallic alloys, or other suitable materials as would be understood by one of skill in the art. The clutch carrier 130 can be configured to have a hollow, cylindrical shape wherein the hollow portion of the clutch carrier 130 is molded to fit the elastic element of the system 100. In the embodiment shown in FIGS. 8A-8E, the bottom of the clutch carrier 130 is configured with indentions, protrusions, splines, castellations, and/or other suitable geometric features as would be understood by one of skill in the art that mate with the corresponding indentations, protrusions, splines, castellations, and/or other geometric features of the clutch disks 110, 120. This is best shown in FIG. 8E. These indentations, protrusions, splines, castellations, and/or other geometric features allow the clutch carrier 130 to sit on top of and engage with the top clutch disk 120, which in turn interfaces with the bottom clutch disk 110. Once the torque limit is reached by the device, the top clutch disk 120 will “slip” with the bottom disk 110, thereby limiting torque transfer and allowing it to apply a precise amount of torque.

At least one advantage of systems and methods incorporating features of the present disclosure is that the design of the torque-limiting slip-clutch system has been modularized and the clutch carrier component has been separated from the slip-clutch disks. Modularization and separation of these components in a slip-clutch assembly not only simplifies the design, but allow for better molding and casting of the clutch disks, resulting in more consistent material properties and geometry. The modular design additionally simplifies sterilization in designs that are not single-use.

Another advantage of the systems and methods incorporating the features of the present disclosure is that the clockwise and counter-clockwise torque can be finely-tuned by adjusting the lobe geometry (e.g., the number of lobes, the width, height, etc.) of the clutch disks. The simplicity of the disks, and thus ease of manufacturing, allows for the use of various materials that can be used to achieve differing and/or higher, more accurate set-points and tolerances. The set-point torque (i.e., the maximum torque or output torque) of the torque-limiting slip-clutch systems 100 disclosed herein can be tuned across a wide range of force. For instance, and not by way of limitation, systems 100 according to the present disclosure can be set to an output torque of 14 newton-meters or above. Other embodiments may be set to 15 newton-meters and above. Still other embodiments may be set to 16 newton-meters or above. Other systems 100 according to the present disclosure can be set to an output torque of less than 14 newton-meters. It will be understood by those in the art that any set-point torque can be selected according the design specifications of a particular application.

It is understood that embodiments presented herein are meant to be exemplary. Embodiments of the present disclosure can comprise any combination of compatible features shown in the various figures, and these embodiments should not be limited to those expressly illustrated and discussed. For instance and not by way of limitation, the appended claims could be modified to be multiple dependent claims so as to combine any combinable combination of elements within a claim set, or from differing claim sets.

Although the present disclosure has been described in detail with reference to certain preferred configurations thereof, other versions are possible. Therefore, the spirit and scope of the disclosure should not be limited to the versions described above.

Additionally, is it understood that the components and concepts in the present disclosure can be applied to devices not specifically mentioned herein. For instance, these components and concepts can be applied to any machinery with rotating components, including without limitation, general machining and manufacturing, conveyors, gearboxes, generators, and motors.

The foregoing is intended to cover all modifications and alternative constructions falling within the spirit and scope of the disclosure as expressed in the appended claims, wherein no portion of the disclosure is intended, expressly or implicitly, to be dedicated to the public domain if not set forth in the claims.

We claim:

1. A torque-limiting slip-clutch system, comprising:
a top clutch disk and an opposing bottom clutch disk, each configured with a plurality of lobes;
an elastic component; and
a clutch carrier configured to receive said elastic component,
wherein said clutch carrier is configured to interface with said top clutch.

2. The torque-limiting slip-clutch system of claim 1, wherein said top clutch disk and said bottom clutch disk each comprise a plurality of lobes.

3. The torque-limiting slip-clutch system of claim 2, wherein said top clutch disk and said opposing bottom clutch disk are each configured with five lobes.

4. The torque-limiting slip-clutch system of claim 2, wherein said top clutch disk and said opposing bottom clutch disk are each configured with six or more lobes.

5. The torque-limiting slip-clutch system of claim 1, wherein said top clutch disk and said opposing bottom clutch disk are modular.

6. The torque-limiting slip-clutch system of claim 1, wherein said top clutch disk is made of a metallic alloy.

7. The torque-limiting slip-clutch system of claim 1, wherein said opposing bottom clutch disk comprises a composite-filled resin material.

8. The torque-limiting slip-clutch system of claim 1, wherein said elastic component comprises a plurality of flat and Belleville washers.

9. The torque-limiting slip-clutch system of claim 1, wherein said elastic component comprises one or more coil springs.

10. The torque-limiting slip-clutch system of claim 8, wherein said washers are configured to stack vertically on top of one another in said clutch carrier.

11. The torque-limiting slip-clutch system of claim 1, wherein said carrier defines a cylindrical housing configured to fit said elastic component with one open end and one closed end.

12. The torque-limiting slip-clutch system of claim 1, wherein said closed end of said clutch carrier is configured with protrusions that mirror the lobe geometry of said top clutch allowing for said clutch carrier to apply torque to said top clutch.

13. The torque-limiting slip-clutch system of claim 1, wherein the output torque of said system is set to fourteen newton-meters.

14. The torque-limiting slip-clutch system of claim 1, wherein the output torque of said system is set to more than fourteen newton-meters.

15. The torque-limiting slip-clutch system of claim 1, wherein said system is disposable.

16. A torque-limiting driver comprising:
a slip-clutch comprising
a top clutch disk and an opposing bottom clutch disk, each configured with a plurality of lobes;
an elastic component;
a clutch carrier configured to receive said elastic component; and
a body comprising an attachment mechanism configured to attach to said slip-clutch,
wherein said top clutch disk is configured to slip with respect to said bottom disk upon the application of a predetermined torque threshold.

17. The torque-limiting driver of claim 16, wherein said predetermined torque threshold is set by said elastic component.

18. The torque-limiting driver of claim 16, wherein said elastic component comprises one or more Bellville washers.

19. The torque-limiting driver of claim 16, wherein said top clutch disk is conical.

20. The torque-limiting driver of claim 19, wherein said bottom clutch disk is conical.
